(19) United States
(12) Patent Application Publication
(10) Pub. No.: US 2008/0207745 A1
(43) Pub. Date: Aug. 28, 2008

(54) Orally-absorbed formulation for paromomycin

(75) Inventors: Gita Natarajan Shankar, Saratoga, CA (US); Helen Jaber Parish, Sunnyvale, CA (US)

Correspondence Address: RICHARD ARON OSMAN, 4070 CALLE ISABELLA, SAN CLEMENTE, CA, 92672, US

(73) Assignee: SRI International

(21) Appl. No.: 11/710,695

(22) Filed: Feb. 24, 2007

(51) Int. Cl. A61K 31/35

(52) U.S. Cl. 514/453

(57) ABSTRACT

A composition for oral treatment of visceral leishmaniasis comprises paromomycin and a permeation enhancer that increases oral bioavailability of the paromomycin to at least 20%.

Statement regarding federally sponsored research: This work was supported by a Federal Grant from the National Institute of Allergy and Infectious Diseases (Contract No. NO1-AI-05414). The U.S. government may have rights in any patent issuing on this application

### BACKGROUND OF THE INVENTION

The field of the invention is oral formulations for paromomycin.

Paromomycin sulfate (PAR) is an antibiotic indicated for intestinal amebiasis and as adjunctive therapy in hepatic coma when given orally. It is poorly absorbed after oral administration, with almost 100% of the drug recoverable in the stool (6). PAR is also used parenterally for treating Visceral Leishmaniasis (VL) in combination with sodium stibogluconate in developing countries. In 2004, an injectable form of PAR was tested in the largest prospective clinical trial ever performed for treatment of VL (1). PAR has also been effective in animal models of multiple-drug resistant tuberculosis (MDR TB) and mycobacterium avium complex (2, 3). However, poor oral absorption of PAR requiring parenteral administration limits its use as a drug of choice for treating these and other infectious diseases, including prophylactic use for opportunistic infections in HIV/AIDS patients (4).

### SUMMARY OF THE INVENTION

One aspect of the invention is a composition for oral treatment of visceral leishmaniasis, said composition comprising: paromomycin; and a permeation enhancer that increases oral bioavailability of the paromomycin, wherein the composition has a paromomycin bioavailability of at least 20% when orally administered to a mammal.

In specific embodiments, the permeation enhancer increases oral bioavailability of the paromomycin by enhancing paracellular transport.

In various embodiments, the permeation enhancer is selected from the group consisting of medium-chain glycerides, macrogolglycerides, polyglycols, sodium caprate, sodium caprylate, and mixtures thereof.

In a specific embodiment the composition comprises 1-20 wt. % caprylocaproyl macrogol-8 glycerides, optionally with 1-10 wt. % d-α-tocopheryl polyethylene glycol 1000 succinate. In specific embodiments, the composition further comprises a Pgp inhibitor, which can be formulated as a shell surrounding the paromomycin.

The composition can be prepared in unit dosage form, such as a liquid or powder form that is filled into capsules that is optionally enteric coated. In another embodiment, the composition is in a liquid form.

In preferred embodiments, the paromomycin bioavailability is at least 30%.

Another aspect of the invention is a method for treating leishmaniasis in a mammal, the method comprising: administering to the mammal a composition comprising: paromomycin; and a permeation enhancer that increases oral bioavailability of the paromomycin, wherein the composition has a paromomycin bioavailability of at least 20% when orally administered to a mammal. In a specific embodiment, prior to the administering step, the mammal is administered a Pgp inhibitor.

A further aspect of the invention is a kit that contains a composition comprising paromomycin and a permeation enhancer that increases oral bioavailability of the paromomycin, wherein the composition has a paromomycin bioavailability of at least 20% when orally administered to a mammal. The kit may further comprise a Pgp inhibitor.

### DETAILED DESCRIPTION OF SPECIFIC EMBODIMENTS OF THE INVENTION

The present invention provides compositions, kits, and methods for oral treatment of visceral leishmaniasis (Kala Azar) and other conditions amenable to systemic treatment with paromomycin. The composition comprises paromomycin, which has a molecular formula of C₂₃H₄₅N₅O₁₄, or pharmaceutically acceptable salts thereof (e.g. sulfate, phosphate, etc.). Paromomycin and salts thereof are referred to herein as paromomycin.

The composition further comprises a permeation enhancer that increases oral bioavailability of the paromomycin to at least 20% when orally administered to a mammal. Enhancers that maximize paromomycin absorption at the jejunum are particularly preferred. Numerous intestinal permeation enhancers and their preferential sites of absorption are known (see Aungst et al, J. Pharm Sci (2000) 89:429-442). The preferential site of absorption of a permeation enhancer/drug combination can also be determined using routine methods, such as the Ussing chamber method (see e.g. Gotoh et al, J Biomol Screen. (2005) 10:517-523; and Example 1 below).

Suitable enhancers are selected from medium-chain glycerides, macrogolglycerides, polyglycols, glycerol esters of fatty acids, pegylated alcoholic esters of fatty acids, glyceryl monoesters, propylene glycol monoesters, medium chain fatty acids, chitosan and chitosan derivatives, and mixtures thereof. The term medium-chain glycerides (MCGs) generally refers to monoglycerides and diglycerides of fatty acid, and may contain triglycerides as well as monoglycerides and diglycerides of shorter and longer chain fatty acids. Examples of preferred MCGs include glyceryl monooleate (oleic acid acyl chain) and glyceryl monolinoleate (linoleic acid acyl chain). Examples of preferred macrogolglycerides are lauroyl macrogol-32 glycerides, sold as GELUCIRE 44/14 (Gattefosse Corporation, Paramus, N.J.) and caprylocaproyl macrogol-8 glycerides, sold as LABRASOL (Gattefosse Corporation, Paramus, N.J.). In a specific embodiment, the composition comprises 1-20 wt. %, and more preferably 2-10 wt. % caprylocaproyl macrogol-8 glycerides.

Preferred combinations of enhancers include mixtures of d-α-tocopheryl polyethylene glycol 1000 succinate (TPGS) (Eastman Vitamin E TPGS, Eastman, Kingsport, Tenn.) with caprylocaproyl macrogol-8 glycerides and/or polysorbates. In a specific embodiment, the composition comprises 1-20 wt. % caprylocaproyl macrogol-8 glycerides and 1-10 wt. % d-α-tocopheryl polyethylene glycol 1000 succinate.

Particularly preferred enhancers are those which enhance paracellular transport. Examples of such enhancers include the medium chain fatty acids caprate, laurate, caprylate, and derivatives thereof (e.g. sodium N-[8-(2-hydroxybenzoyl)amino]caprylate; see e.g. Hess et al., Eur J Pharm Sci. (2005) 25:307-12); and chitosan and chitosan derivatives (see e.g. Cano-Cebrian et al., Curr Drug Deliv. (2005) 2:9-22). Sodium caprate (also known as sodium decanoate), and sodium caprylate are particularly preferred enhancers.

The permeation enhancer in the composition is provided in an effective amount, preferably at or above but proximate to a minimal effective amount, preferably less than about 80% by weight (wt. %) of the composition. More preferably, the amount of intestinal permeation enhancer is less than 60, 50, 40 or 30 wt. %. The amount of paromomycin in the composition is preferably at least 20% by weight of the composition. More preferably, the composition comprises at least 30, 40, 50, 60, 70, or 80 wt % of the paromomycin. In some embodiments, the composition consists essentially of the paromomycin and the permeation enhancer, particularly in the case of liquid formulations. In other embodiments, the composition comprises additional excipients, binders, etc. for example to facilitate formulation into tablets, pellets, beads, etc.

The particular form of the composition (i.e. solution, viscous gel, powder, tablet, etc.) will generally be dictated by the selected permeation enhancer. As one example, the composition can be prepared as a liquid formulation, suitable for drinking or filling into capsules, by dissolving paromomycin in HEPES buffer containing 5 wt. % Labrasol® and 5 wt. % Vitamin E TPGS. As another example, the composition can be prepared as a powder by mixing paromomycin with sodium caprate. The powder can then be combined with binders and pressed into tablets, or filled into capsules. Unit dosage forms (e.g. tablets, capsules, etc.) contain from about 5-450 mg of paromomycin, and are optionally enteric coated using known methods.

Intestinal epithelial cells express transport systems that actively remove certain compounds from the cells, transporting them in the blood-to-lumen direction. We have found that these transport systems are operative against paromomycin. Accordingly, the composition may further comprise a secretory transport inhibitor. In specific embodiments, the transport inhibitor is a Pgp inhibitor such as Vitamin E TPGS, quinidine, digoxin, and verapamil. Numerous other suitable Pgp inhibitors are well-known (see e.g. Aungst, supra). The Pgp inhibitor may be administered as a predose in a liquid or solid form at a specified time interval (e.g. 5, 10, 15, 30, 60 minutes) prior to administration of the paromomycin. Alternatively, the Pgp inhibitor may be co-formulated with the paromomycin. In one co-formulation, the Pgp inhibitor, paromomycin, and enhancer are combined in a homogenous mixture or solution that can be filled into capsules. In another embodiment, the Pgp inhibitor is included as a solid in a solid/liquid matrix comprising the paromomycin and enhancer. In yet another formulation, the paromomycin and enhancer are formulated in a matrix with an outer sheathing of the Pgp inhibitor present as an outside shell. The capsule with the inner core of paromomycin/enhancer and the outer layer of Pgp inhibitor can be optionally coated by an enteric coating to promote delivery of both the inner core of drug and the outer covering of Pgp inhibitor at the target site.

The compositions are formulated such that the bioavailability of the paromomycin when orally administered to a mammal is at least 20%, and is preferably at least 30, 40, or 50%. Oral bioavailability can be assessed using the Beagle dog, or equivalent model, wherein levels of plasma paromomycin from an orally administered composition are compared to levels obtained after i.v. administration using the calculation: BA=AUC_{PO}×Doseᵢᵥ/AUCᵢᵥ×Dose_{PO}; where BA=bioavailability and AUC=area under the plasma concentration-time curve.

The above-described composition is orally administered to a mammal that has a condition amenable to systemic treatment with paromomycin, such as visceral leishmaniasis (Kala Azar). In specific embodiments, the mammal is a human. In other embodiments, the mammal may be a livestock animal (horse, cow, pig, etc.) or a companion animal (e.g. dog, cat, etc.). For humans, the daily dose of absorbed paromomycin for effective treatment of visceral leishmaniasis is approximately 20 mg/kg. Thus, a composition of the invention that has a paromomycin bioavailability of approximately 50% will be administered at a dose of approximately 40 mg/kg/day paromomycin.

In certain embodiments, a separately formulated Pgp inhibitor is administered prior to or together with the DTPA chelate composition. The composition may be provided in a kit with instructions on proper dosing. For example, the composition may be provided in a blister-pack kit, where one or more unit dosage forms are contained in a blister. The blister packaging may contain writing adjacent a blister or a row or column of blisters to indicate the proper timing of dosing. The kit may additionally contain a separately formulated Pgp inhibitor.

### EXAMPLE 1

### In Vitro Drug Transport Studies and Sample Analysis

PAR transport across representative sections of harvested rat small intestinal segments and colon was initially measured in-vitro using an EasyMount Ussing System (Physiological Instruments Inc., CA, Item # EM-CSYS-8). To investigate the causes of poor oral absorption of PAR, both mucosal to serosal (M-to-S) transport and serosal to mucosal (S-to-M) transport across harvested rat jejunum segment was measured.

PAR was dissolved in HEPES buffer (135 mM NaCl, 5.4 mM KCl, 1.2 mM CaCl₂, 1.2 mM MgCl₂, 5.0 mM NaOH, 10 mM HEPES, 10 mM dextrose), with or without additives, to obtain the predetermined drug concentrations. HEPES buffer was used as the base mucosal and serosal fluid. Transport of PAR across representative sections of the harvested rat small intestine (i.e. jejunum, duodenum, and ileum) and colon was monitored by mounting the harvested segments on sliders placed between the two chambers. First 5 ml of HEPES buffer was added to each of the two chambers (mucosal and serosal) and allowed to equilibrate for 20 min. Then the simple HEPES buffer either mucosal or serosal chamber was replaced by HEPES buffer containing PAR and appropriate formulation additives to be tested (e.g. 5 mg/ml of PAR dissolved in HEPES buffer containing 10% w/w Labrasol®). Aliquots of sample solution were removed from the chamber on other side of the mounted intestinal segment periodically and replaced with an equal volume of fresh warm HEPES buffer.

The effects of different additives, i.e. permeation enhancers, P-glycoprotein (Pgp) substrate and/or inhibitors on transport of PAR across different segments of intestine and colon were also determined using the Ussing system. These additives, either alone or in combinations, were dissolved in HEPES buffer on percent weight/weight basis of the HEPES medium. Owing to the expected variation in the in-vitro conditions and harvested rat intestinal segments, four replicates were used per experimental condition.

The apparent permeability coefficients (Pₐₚₚ) were calculated according to:

Pₐₚₚ=(dQ/dt)/C₀×A, where dQ/dt is the linear appearance rate of mass in receiver compartment, C₀ is the initial solute concentration in donor compartment, and A is the surface area.

A modified HPLC method was used for analyzing PAR samples from in-vitro transport studies (5). The reverse phase method with a Zorbax sb C18 column, 5 μm, 250×4.6 mm (maintained at 50° C.) mobile phase 64% methanol and 36% water at a flow rate of 1.5 ml/min was used for quantifying the drug substance. The drug in post column eluant was derivatized with Dinitrofluorobenzene (DNFB) and monitored at 360 nm. The run time was 30 minutes.

PAR transport into serosal chamber, based on calculated Pₐₚₚ values, was highest for jejunum and lowest for duodenum. Cumulative mean concentrations of PAR released into the serosal side as a function of time is shown in Table 1. Pₐₚₚ values for PAR transport are given in Table 2.

TABLE 1
Amount of PAR in HEPES buffer (pH 7.4) transported across
intestinal segments

| | Average (Ave) Cumulative Amount of | | | | | | | |
|---|---|---|---|---|---|---|---|---|
| | Paromomycin Released (μg) (5 mg/ml or | | | | | | | |
| | 25 mg total of Paromomycin in HEPES Buffer); | | | | | | | |
| | Exposed tissue area = 0.5 sq cm | | | | | | | |
| Time | Duodenum | | Jejunum | | Ileum | | Colon | |
| (hr) | Ave | Std dev | Ave | Std dev | Ave | Std dev | Ave | Std dev |
| 0.5 | 0.00 | 0.00 | 0.00 | 0.00 | 0.00 | 0.00 | 0.00 | 0.00 |
| 1 | 0.00 | 0.00 | 0.00 | 0.00 | 0.00 | 0.00 | 0.00 | 0.00 |
| 1.5 | 10.50 | 1.64 | 26.81 | 2.96 | 14.30 | 2.24 | 8.63 | 6.91 |
| 2 | 20.51 | 2.06 | 44.27 | 5.77 | 25.23 | 4.34 | 21.81 | 8.60 |
| 2.5 | 33.30 | 1.37 | 66.25 | 5.66 | 37.40 | 6.74 | 35.67 | 10.59 |

TABLE 2
Pₐₚₚ Comparisons Across Various Intestinal Segments

| HEPES Buffer with 5 mg/ml | | |
|---|---|---|
| (~25 mg) of paromomycin in | | |
| mucosal chamber; Exposed | | Std Dev |
| tissue area = 0.5 sq cm | Pₐₚₚ (×10⁻⁶ cm/sec) | (×10⁻⁶) |
| Duodenum | 1.94 | 0.30 |
| Jejunum | 3.93 | 0.53 |
| Ileum | 2.22 | 0.30 |
| Colon | 2.07 | 0.36 |

Increasing the concentration of drug in the mucosal chamber resulted in an increase in M-to-S transport of PAR in a dose-dependent manner, as shown in Table A.

TABLE A
Table A

| | Paromomycin: Average (Ave) Cumulative | | | | | | | |
|---|---|---|---|---|---|---|---|---|
| | Amount of Paromomycin Released (μg) | | | | | | | |
| | (X mg/ml of Paromomycin in HEPES Buffer); | | | | | | | |
| | Exposed tissue area = 0.5 sq cm | | | | | | | |
| | X = 5 mg/ml | | X = 15 | | X = 50 | | | |
| Time | | Std | mg/ml | | mg/ml | | X = 75 mg/ml | |
| (hr) | Ave | dev | Ave | Std dev | Av | Std dev | Ave | Std dev |
| 0.5 | 0.0 | 0.0 | 0.0 | 0.0 | 40.8 | 29.6 | 48.6 | 22.5 |
| 1 | 0.0 | 0.0 | 24.1 | 2.9 | 155.9 | 66.8 | 235.2 | 82.6 |
| 1.5 | 26.8 | 3.0 | 52.9 | 5.5 | 284.5 | 103.5 | 495.7 | 60.1 |
| 2 | 44.3 | 5.8 | 94.6 | 8.0 | 468.5 | 189.5 | 776.5 | 46.1 |
| 2.5 | 66.2 | 5.7 | 142.5 | 11.2 | 680.0 | 225.1 | 1104.6 | 107.4 |

Different permeation enhancers, i.e. Labrasol®, Gelucire® 44/14, Vitamin E TPGS® and surfactants such as Polysorbate-80; either alone or in combination were evaluated for their effect on in-vitro transport of PAR from M-to-S side. Among the permeation enhancers used in this study, Labrasol® alone at low concentration of 5% w/w had maximum permeation enhancement. The results are presented in Table B and C. Pₐₚₚ values for PAR transport, in the presence of different enhancers, are given in Table 3.

TABLE B

| | Average Cumulative Amount of Paromomycin | | | | | | | |
|---|---|---|---|---|---|---|---|---|
| | Released (μg) (HEPES Buffer | | | | | | | |
| | with 15 mg/ml of Paromomycin and | | | | | | | |
| | enhancers); Exposed tissue | | | | | | | |
| | area = 0.5 sq cm | | | | | | | |
| | | | | | | | 5% w/w | |
| | | | | | 5% w/w | | Vitamin E | |
| | | | 5% w/w | | Vitamin E | | TPGS, 5% w/w | |
| Time | HEPES buffer | | Labrasol | | TPGS | | Labrasol | |
| (hr) | Ave | Std Dev | Ave | Std Dev | Ave | Std Dev | Ave | Std Dev |
| 0.5 | 0.0 | 0.0 | 10.3 | 9.6 | 2.5 | 5.6 | 1.3 | 3.1 |
| 1 | 24.1 | 2.9 | 59.9 | 7.3 | 22.3 | 18.0 | 27.7 | 7.6 |
| 1.5 | 52.9 | 5.5 | 120.2 | 13.0 | 55.8 | 27.0 | 69.2 | 14.6 |
| 2 | 94.6 | 8.0 | 192.8 | 19.9 | 103.8 | 40.3 | 125.8 | 22.0 |
| 2.5 | 142.5 | 11.2 | 277.4 | 30.9 | 163.7 | 55.4 | 192.6 | 32.6 |

TABLE C

| | Average Cumulative Amount of Paromomycin | | | | | | | |
|---|---|---|---|---|---|---|---|---|
| | Released (μg) (HEPES Buffer | | | | | | | |
| | with 15 mg/ml of Paromomycin and | | | | | | | |
| | enhancers); Exposed tissue area = 0.5 sq cm | | | | | | | |
| | | | | | | | 5% w/w | |
| | | | | | | | Tween | |
| | | | 5% w/w | | 20% w/w | | 80 + 5% w/w | |
| Time | HEPES buffer | | Labrasol | | bile salt | | Captex 355 | |
| (hr) | Ave | Std Dev | Ave | Std Dev | Ave | Std Dev | Ave | Std Dev |
| 0.5 | 0.0 | 0.0 | 3.3 | 6.7 | 0.0 | 0.0 | 0.0 | 0.0 |
| 1 | 24.1 | 2.9 | 32.6 | 9.3 | 19.7 | 17.1 | 15.9 | 3.4 |
| 1.5 | 52.9 | 5.5 | 60.0 | 11.7 | 70.8 | 25.0 | 43.3 | 8.2 |
| 2 | 94.6 | 8.0 | 98.6 | 17.2 | 126.9 | 33.6 | 84.5 | 14.1 |
| 2.5 | 142.5 | 11.2 | 163.3 | 32.5 | 190.7 | 41.6 | 121.3 | 18.6 |

TABLE 3
PAR Transport (Pₐₚₚ values) Across the Jejunum in Presence of
Different Permeation Enhancers

| Transport in Jejunum Segments | | |
|---|---|---|
| 15 mg/ml of PAR in HEPES buffer | Paromomycin | |
| containing enhancers. Exposed tissue | | Std Dev |
| area = 0.5 sq cm | Pₐₚₚ (×10⁻⁶ cm/sec) | (×10⁻⁶) |
| only HEPES buffer | 2.63 | 0.22 |
| 5% (w/w) Labrasol ® | 4.94 | 0.30 |
| 5% (w/w) Vitamin E TPGS ® | 2.99 | 0.34 |
| 5% (w/w) Vitamin E TPGS ® + 5% | 3.56 | 0.35 |
| (w/w) Labrasol ® | | |
| 5% (w/w) Polysorbate 80 + 5% (w/w) | 2.89 | 0.23 |
| Labrasol ® | | |
| 5% (w/w) Gelucire ® 44/14 + 5% | 2.86 | 0.32 |
| (w/w) Labrasol ® | | |
| 20% (w/w) Bile salt | 3.62 | 0.36 |
| 5% (w/w) Polysorbate 80 + 5% (w/w) | 2.31 | 0.21 |
| Captex 355 | | |

Increasing the concentration of PAR in the mucosal chamber resulted in an increase in the M-to S transport (Papp values) in a dose-dependent manner even in the presence of enhancers (5% w/w Vitamin E TPGS®+5% w/w Labrasol®), as shown in Table D and Table 4.

TABLE D

| | Average Cumulative Amount Release (μg) (X mg/ml of | | | | | |
|---|---|---|---|---|---|---|
| | Paromomycin in HEPES Buffer containing 5% w/w | | | | | |
| | Vitamin E TPGS + 5% w/w Labrasol) | | | | | |
| Time | X = 15 mg/ml | | X = 50 mg/ml | | X = 100 mg/ml | |
| (hr) | Average | Std Dev | Average | Std Dev | Average | Std Dev |
| 0.5 | 1.26 | 3.10 | 10.89 | 3.25 | 57.33 | 26.17 |
| 1 | 27.66 | 7.63 | 65.61 | 18.40 | 267.65 | 105.42 |
| 1.5 | 69.24 | 14.57 | 142.86 | 48.06 | 555.38 | 181.11 |
| 2 | 125.77 | 22.04 | 261.53 | 68.01 | 943.28 | 205.27 |
| 2.5 | 192.59 | 32.58 | 505.68 | 51.62 | 1531.18 | 475.51 |

TABLE 4
Pₐₚₚ Comparisons of Transport Across Different Intestinal Segments
for PAR with Permeation Enhancers

| X mg/ml of PAR in HEPES | Paromomycin | |
|---|---|---|
| Buffer with 5% (w/w) Vitamin | | Std Dev |
| E TPGS ® + 5% (w/w) Labrasol ® | Pₐₚₚ (×10⁻⁶ cm/sec) | (×10⁻⁶) |
| x = 15 | 3.56 | 0.35 |
| x = 50 | 2.63 | 0.48 |
| x = 100 | 4.03 | 0.47 |

The results from studies evaluating the effect of Quinidine, a Pgp inhibitor added to serosal and mucosal chambers on the M-to-S transport of PAR; and in the presence or absence of permeation enhancers is presented in Table 5. Quinidine added to the mucosal chamber enhanced M-to-S transport of PAR. In presence of permeation enhancers, i.e. 5% w/w Vitamin E TPGS®+5% w/w Labrasol®, higher Pₐₚₚ values were obtained at 0.5 mg/ml Quinidine concentration, when compared to 0.05 mg/ml Quinidine concentration. Polysorbate-80 has shown a slightly negative effect on M-to-S transport (Pₐₚₚ values) of PAR in presence of Quinidine.

TABLE 5
Table 5: Pₐₚₚ Values for “Transport” Across Jejunum Segments -
Comparisons Between PAR With and Without
Quinidine (Pgp Inhibitor)

| | | Paromomycin | |
|---|---|---|---|
| 15 mg/ml of PAR in | | Pₐₚₚ | |
| HEPES Buffer with and without | | (×10⁻⁶ | Std Dev |
| Additives; Exposed tissue area = 0.5 sq cm | | cm/sec) | (×10⁻⁶) |
| HEPES Buffer | No Quinidine | 2.63 | 0.22 |
| | 0.05 mg/ml Quinidine | 4.37 | 0.24 |
| | in mucosal | | |
| HEPES Buffer containing | No Quinidine | 4.94 | 0.30 |
| 5% w/w Labrasol ® | 0.05 mg/ml Quinidine | 5.00 | 0.43 |
| | in mucosal | | |
| HEPES Buffer containing | No Quinidine | 3.56 | 0.35 |
| 5% w/w Vitamin E | 0.5 mg/ml Quinidine | 7.20 | 0.50 |
| TPGS ® + 5% w/w | in mucosal. (1^{st} study) | | |
| Labrasol ® | 0.5 mg/ml Quinidine | 5.53 | 0.28 |
| | in mucosal. (2^{nd} study) | | |
| | 0.05 mg/ml Quinidine | 4.23 | 0.45 |
| | in serosal | | |
| HEPES Buffer containing | No Quinidine | 2.89 | 0.23 |
| 5% w/w Polysorbate 80 + | 0.05 mg/ml Quinidine | 2.79 | 0.28 |
| 5% w/w Labrasol ® | in mucosal | | |
| HEPES Buffer containing | No Quinidine | 2.86 | 0.32 |
| 5% w/w Polysorbate 80 + | 0.05 mg/ml Quinidine | 2.55 | 0.24 |
| 5% w/w Labrasol ® | in mucosal | | |
| HEPES Buffer containing | 0.05 mg/ml Quinidine | 5.00 | 0.43 |
| 5% w/w Labrasol ® | in mucosal | | |
| HEPES Buffer containing | 0.05 mg/ml Quinidine | 3.50 | 0.42 |
| 15% w/w Labrasol ® | in mucosal | | |
| HEPES Buffer containing | 0.05 mg/ml Quinidine | 5.83 | 1.39 |
| 40% w/w Labrasol ® | in mucosal | | |

Table 6 gives the Pₐₚₚ values for PAR, in the absence of additives, in the M-to-S as well as S-to-M direction to be 2.63+/−0.22×10⁻⁶ cm/sec and 2.75+/−0.22×10⁻⁶ cm/sec, respectively. This is suggestive of the presence of efflux mechanisms for PAR. The net effective transport can be influenced by the presence of enhancers and Pgp inhibitors, as seen in Table 6. HEPES Buffer containing 5% w/w Vitamin E TPGS®+5% w/w Labrasol® and 0.5 mg/ml Quinidine in mucosal gave the highest transport value for PAR in the M-to-S direction.

TABLE 6
Pₐₚₚ Values for “Transport” of PAR in both
M-to-S and S-to-M Directions

| | | Paromomycin | |
|---|---|---|---|
| 15 mg/ml (total 75 mg) of | | | Std |
| PAR in mucosal chamber | | Pₐₚₚ | Dev |
| Exposed tissue area = 0.5 sq cm | | (×10⁻⁶ cm/sec) | (×10⁻⁶) |
| M-to-S | HEPES Buffer only | 2.63 | 0.22 |
| Transport | HEPES Buffer containing 0.05 | 4.37 | 0.24 |
| | mg/ml Quinidine in mucosal | | |
| | HEPES Buffer containing (5% w/w | 7.20 | 0.50 |
| | Vitamin E TPGS ® + 5% | | |
| | w/w Labrasol ®) and 0.5 mg/ml | | |
| | Quinidine in mucosal. | | |
| S-to-M | HEPES Buffer only | 2.75 | 0.22 |
| Transport | HEPES Buffer containing 0.05 | 1.85 | 0.19 |
| | mg/ml Quinidine in mucosal | | |
| | HEPES Buffer containing (5% w/w | 1.30 | 0.21 |
| | Vitamin E TPGS ® + 5% | | |
| | w/w Labrasol ®) and 0.5 mg/ml | | |
| | Quinidine in mucosal. | | |

The results of in vitro transport studies using sodium decanoate, added to mucosal and serosal chambers in the presence or absence of enhancer are presented in Tables 7 and 8. Sodium decanoate added to the serosal chamber at higher concentrations of 1 mg/ml resulted in appreciable increase in M-to-S transport of Paromomycin.

TABLE 7
Pₐₚₚ Values for “Transport” Across Jejunum
Segments - comparisons Between PAR With and Without
Sodium Decanoate in the Absence of Other Permeation Enhancers

| HEPES Buffer with 15 mg/ml | | Paromomycin | |
|---|---|---|---|
| (total 75 mg) of Paromomycin; | | Pₐₚₚ | Std Dev |
| Exposed tissue area = 0.5 sq cm | | (×10⁻⁶ cm/sec) | (×10⁻⁶) |
| Sodium | No Sodium Decanoate | 2.63 | 0.22 |
| Decanoate in | 0.2 mg/ml (equilibrated)* | 1.48 | 0.24 |
| Mucosal | 0.5 mg/ml (equilibrated) | 2.22 | 0.14 |
| | 1 mg/ml (equilibrated) | 4.40 | 0.33 |
| | 1 mg/ml (not equilibrated) | 3.83 | 0.24 |
| Sodium | No Sodium Decanoate | 2.63 | 0.22 |
| Decanoate in | 0.2 mg/ml (not equilibrated) | 3.86 | 0.34 |
| Serosal | 0.5 mg/ml (not equilibrated) | 3.54 | 0.39 |
| | 1.0 mg/ml (not equilibrated) | 7.20 | 0.58 |

*Equilibration is done by adding Sodium Decanoate 20 minutes prior to adding the drug

TABLE 8
Pₐₚₚ Values for “Transport” Across Jejunum
Segments - Comparisons Between PAR With and Without
Sodium Decanoate in the Presence of Other Permeation Enhancers

| HEPES Buffer with 15 mg/ml of Paromomycin | | Paromomycin | |
|---|---|---|---|
| in 5% w/w Vitamin E TPGS ® + 5% w/w | | Pₐₚₚ | Std Dev |
| Labrasol ® Exposed tissue area = 0.5 sq cm | | (×10⁻⁶ cm/sec) | (×10⁻⁶) |
| Sodium | No Sodium Decanoate | 3.56 | 0.35 |
| Decanoate in | 0.2 mg/ml (equilibrated*) | 3.99 | 0.17 |
| mucosal | 0.5 mg/ml (equilibrated) | 2.43 | 0.10 |
| | 1.0 mg/ml (equilibrated) | 2.55 | 0.12 |
| | No Sodium Decanoate | 3.56 | 0.35 |
| Sodium | 0.2 mg/ml (equilibrated) | 4.86 | 0.39 |
| Decanoate in | 0.2 mg/ml (not equilibrated) | 2.01 | 0.13 |
| serosal | 0.5 mg/ml (not equilibrated) | 1.94 | 0.10 |
| | 1.0 mg/ml (not equilibrated) | 6.01 | 0.46 |

*Equilibration is done by adding sodium decanoate 20 minutes prior to adding the drug.

### EXAMPLE 2

### Paromomycin In Vivo Absorption Studies in Rats

Male rats (Sprague Dawley) were randomly divided into four groups with each group having three animals and were administered with PAR, either as an oral composition at a dose of 1000 mg/kg or an injection at a dose of 100 mg/kg dissolved in normal saline. Three different oral formulation compositions selected based on the in vitro transport studies were used in the in vivo absorption studies in rat animal model. Blood samples were collected and processed to obtain plasma at the following time points: intravenous (iv) dose group at 5, 10, 20, 30 min, and further at 1, 2, 3, 9, and 24 hr; oral dose groups at 15, and 30 min, and then at 1, 2, 4, 6, 8, 10, and 24 hr time-points after dose administration.

Paromomycin was extracted from plasma samples (100 μl volume) with phenylboronic acid solid phase extraction columns, and the bound drug was eluted from the columns with 1% (v/v) formic acid in water. The acid eluates were evaporated under vacuum, and then the dry residues were reconstituted in 100 μl of 1 mM heptafluorobutyric acid (HFBA) in water. The samples were then analyzed by LCMS using a C18 reversed phase column and a gradient from water to acetonitrile (both mobile phase constituents containing 1 mM HFBA), and detected by single ion recording on a Micromass Quattro LC triple quadrupole mass spectrometer set to monitor the M+2 ion of paromomycin at m/z 308.7.

Experimental design for in-vivo studies is presented in Table 9. The plasma blood levels observed in the orally administered rats is shown in Table E. The serum drug levels for intravenous and oral groups are given in Tables 10 and 11, respectively.

TABLE 9
Pharmacokinetic study design for paromomycin formulations:
intravenous and oral

| | Route/ | Vehicle | Dose level | Dose conc. | No. of |
|---|---|---|---|---|---|
| Group^{a} | Formulation | (% w/w in HEPES) | (mg/kg) | (mg/ml) | Rats |
| 1 | IV | Sterile saline | 100 | 10 | 3 M |
| 2 | Oral | 5% Labrasol ® and 5% Vitamin E | 1000 | 100 | 3 M |
| | | TPGS ® | | | |
| 3 | Oral | 5% Labrasol ® and 5% Vitamin E | 1000 | 100 | 3 M |
| | | TPGS ® + 10 mM sodium | | | |
| | | decanoate^{a} | | | |
| 4 | Oral | 5% Labrasol ® + 5% Polysorbate | 1000 | 100 | 3 M |
| | | 80 | | | |

^{a}Sodium decanoate was administered orally 30 minutes prior to oral administration of Paromomycin.

TABLE E
Table E

| | Average (Ave) Cumulative Amount | | | | | |
|---|---|---|---|---|---|---|
| | Released (μg) (HEPES Buffer | | | | | |
| | containing X mg/ml of paromomycin | | | | | |
| | and 5% w/w Vitamin E TPGS + 5% | | | | | |
| | w/w Labrasol) | | | | | |
| Time | X = 15 mg/ml | | X = 50 mg/ml | | X = 100 mg/ml | |
| (hr) | Ave | Std Dev | Av | Std Dev | Ave | Std Dev |
| 0.5 | 1.26 | 3.10 | 10.89 | 3.25 | 57.33 | 26.17 |
| 1 | 27.66 | 7.63 | 65.61 | 18.40 | 267.65 | 105.42 |
| 1.5 | 69.24 | 14.57 | 142.86 | 48.06 | 555.38 | 181.11 |
| 2 | 125.77 | 22.04 | 261.53 | 68.01 | 943.28 | 205.27 |
| 2.5 | 192.59 | 32.58 | 505.68 | 51.62 | 1531.18 | 475.51 |

TABLE 10
Pharmacokinetic Analysis for PAR in Male Rats Dosed i.v. (100 mg/mL)
Group # 1

| | t_{1/2} | | | V | |
|---|---|---|---|---|---|
| Rat # | (hr) | Cmax (ng/ml) | AUC (hr · ng/ml) | (L/kg) | Cl (L/hr/kg) |
| 1 | 2.85 | 173931 | 93533 | 4.39 | 1.07 |
| 2 | 2.70 | 95651 | 55465 | 7.01 | 1.80 |
| 3 | 2.86 | 101318 | 85847 | 4.80 | 1.16 |
| Mean | 2.80 | 123633 | 78282 | 5.40 | 1.34 |
| SD | 0.09 | 43651 | 20130 | 1.41 | 0.40 |

TABLE 11
Pharmacokinetic Analysis for PAR in Male Rats Dosed Orally (1000 mg/mL)

| | | | | | | | |
|---|---|---|---|---|---|---|---|
| | | Tmax | Cmax | AUC | V | Cl/F | |
| Formulation | Rat # | (hr) | (ng/ml) | (hr · ng/ml) | (L/kg) | (L/hr/kg) | F (%) |

Group #2

| | | | | | | | |
|---|---|---|---|---|---|---|---|
| 5% w/w | 4 | 1.0 | 137827 | 276965 | 3.44 | 0.72 | 20.10 |
| Labrasol | 5 | 1.0 | 172924 | 344686 | 2.43 | 0.72 | 25.01 |
| 5% w/w | 6 | 1.0 | 371346 | 676064 | 0.96 | 0.73 | 49.05 |
| Vitamin E | Mean | 1.00 | 227366 | 432572 | 2.28 | 0.73 | 31.39 |
| TPGS ® | SD | 0.00 | 125919 | 213572 | 1.25 | 0.00 | 15.50 |

Group #3

| | | | | | | | |
|---|---|---|---|---|---|---|---|
| 5% w/w | 7 | 0.5 | 80925 | 124258 | 7.53 | 0.72 | 9.02 |
| Labrasol | 8 | 1.0 | 39958 | 72956 | 4.41 | 0.72 | 5.29 |
| 5% w/w | 9 | 0.3 | 56299 | 83462 | 14.72 | 0.66 | 6.06 |
| Vitamin E | Mean | 0.58 | 59061 | 93559 | 8.89 | 0.70 | 6.79 |
| TPGS ® + 10 mM | SD | 0.38 | 20623 | 27100 | 5.28 | 0.03 | 1.97 |
| Sodium | | | | | | | |
| Decanoate | | | | | | | |

Group #4

| | | | | | | | |
|---|---|---|---|---|---|---|---|
| 5% w/w | 10 | 0.5 | 394550 | 497665 | 16.53 | 0.72 | 36.11 |
| Labrasol + 5% w/w | 11 | 0.5 | 132022 | 161879 | 4.66 | 0.76 | 11.75 |
| Polysorbate | 12 | 1.0 | 44004 | 95538 | 4.84 | 0.48 | 6.93 |
| 80 | Mean | 0.67 | 190192 | 251694 | 8.68 | 0.65 | 18.26 |
| | SD | 0.29 | 182369 | 215584 | 6.80 | 0.15 | 15.64 |

The formulation additives, i.e. the permeation enhancers, Pgp inhibitors and inducer of tight junctions opening, were well-tolerated by the rats at the given dose in this single dose studies. The bioavailability was also very good, especially because the oral bioavailability of this drug is historically considered to be close to zero (6).

We have determined that PAR may be formulated as an oral dosage form with substantial systemic absorption for treating infections caused by susceptible microorganisms by co-formulating with combinations of permeation enhancers, Pgp inhibitors and/or inducers of tight junctions opening in the intestinal tract. The role Pgp plays in drug absorption and disposition has been studied. (7). Sodium decanoate has been used to improve bioavailability of certain drugs (8).

We have determined that the oral absorption of PAR may be substantially improved in presence of permeation enhancers such as Labrasol® and Vitamin E TPGS®.

### EXAMPLE 2

### Oral Paromomycin Clinical Study

This study assesses the efficacy and tolerability of oral paromomycin compared with injectable paromomycin for treating visceral leishmaniasis and is adapted from a previously published trial (Jha et al, BJM (1998) 316:1200-1205).

Design: Randomized, unblinded, controlled trial with 180 days follow-up.

Setting: Kala-Azar Research Centre, Brahmpura, Muzaffarpur, Bihar, India.

Subjects: 120 patients of either sex aged 6-50 years with symptoms and signs suggestive of visceral leishmaniasis (fever, loss of appetite, enlarged spleen) with leishmania amastigotes detected in Giemsa stained aspirates of spleen or bone marrow.

Interventions: Injectable paromomycin at 20 mg/kg administered intramuscularly for 21 days and oral paromomycin at 25, 50, and 100 mg/kg/day for 21 days (30 patients per treatment arm).

Main outcome measures: Laboratory measures of efficacy: parasite count, haemoglobin concentration, white cell count, platelet count, serum albumin concentration. Clinical measures of efficacy: spleen size, fever, body weight, and liver size. Measures of safety: liver and renal function tests, reports of adverse events.

Results: Cure rates of greater than 90% at end of follow-up demonstrate efficacy of oral paromomycin comparable to that of injectable paromomycin.

### REFERENCES

1. Strom S., A small charity takes lead in fighting a disease, New York Times, Jul. 31, 2006
2. Kanyok et al., Pharmaokinetics of intramuscularly administered aminosidine in healthy subjects, Antimicrob Agents Chemother., 1997 May, 41(5); 982-6
3. T. P. Kanyok, M. V. Reddy, J. Chinnaswamy, L. H. Danziger and P. R. J. Gangadharam. In vivo activity of paromomycin against susceptible and multidrug-resistant Mycobacterium tuberculosis and M. avium complex strains. Antimicrob Agents Chemother. (1994) 38:170-173.
4. F. Bissuel, L. Cotte, M. De Montclos, M. Rabodonirina and C. Trepo. Absence of systemic absorption of oral paromomycin during long-term, high-dose treatment for cryptosporidiosis in AIDS [5]. J Infect Dis. (1994) 170:749-750.
5. Lu J., Cwik M. and Kanyok T., Determination of paromomycin in human plasma and urine by reverse-phase HPLC using 2,4-Dinitrofluorobenzene derivatization, J of Chromatography B Biomed Sci Appl. (1997) 695:329-335
6. Humatin® product information, King Pharmaceuticals.
7. J. H. Lin, M. Yamazaki. Role of P-Glycoprotein in pharmacokinetics: Clinical implications, Clinical Pharmacokinetics. (2003) 42 (1), pp. 59-98.
8. Baluom et al., Synchronized release of sulpiride and Sodium decanoate from HPMC matrices: a rational approach to enhance sulpiride absorption in the rat intestine, Pharmaceutical Research (2000) 17:1071-1076.

What is claimed is:

1. A composition for oral treatment of visceral leishmaniasis, said composition comprising:
paromomycin; and
a permeation enhancer that increases oral bioavailability of the paromomycin, wherein the composition has a paromomycin bioavailability of at least 20% when orally administered to a mammal.

2. The composition of claim 1 wherein the permeation enhancer increases oral bioavailability of the paromomycin by enhancing paracellular transport.

3. The composition of claim 1 wherein the permeation enhancer is sodium caprate.

4. The composition of claim 1 wherein the permeation enhancer is selected from the group consisting of medium-chain glycerides, macrogolglycerides, polyglycols, sodium caprate, sodium caprylate, and mixtures thereof.

5. The method of claim 1 wherein the permeation enhancer comprises caprylocaproyl macrogol-8 glycerides.

6. The method of claim 1 wherein the composition comprises 1-20 wt. % caprylocaproyl macrogol-8 glycerides.

7. The method of claim 1 wherein the permeation enhancer comprises caprylocaproyl macrogol-8 glycerides and d-α-tocopheryl polyethylene glycol 1000 succinate.

8. The method of claim 1 wherein the composition comprises 1-20 wt. % caprylocaproyl macrogol-8 glycerides and 1-10 wt. % d-α-tocopheryl polyethylene glycol 1000 succinate.

9. The composition of claim 1 further comprising a P glycoprotein (Pgp) inhibitor.

10. The composition of claim 1 further comprising a Pgp inhibitor, wherein the Pgp inhibitor is formulated as a shell surrounding the paromomycin.

11. The composition of claim 1 in an enteric-coated unit dosage form.

12. The composition of claim 1 in a liquid form.

13. The composition of claim 1 in a powder form filled in a capsule.

14. The composition of claim 1 wherein the paromomycin bioavailability is at least 30%.

15. A method for treating leishmaniasis in a mammal, the method comprising:
administering to the mammal the composition of claim 1.

16. The method of claim 15 wherein prior to the administering step, the mammal is administered a Pgp inhibitor.

17. A kit comprising the composition of claim 1.

18. The kit of claim 17 further comprising a Pgp inhibitor.
